(19) United States
(12) Patent Application Publication
(10) Pub. No.: US 2025/0235843 A1
(43) Pub. Date: Jul. 24, 2025

(54) MICROSPHEROIDS CONTAINING DISTINGUISHABLE MICROPARTICLE PATTERNS

(71) Applicant: Droplet Genomics UAB, Vilnius (LT)

(72) Inventors: Karolis Leonavicius, Vilnius (LT); Rokas Stanislovas, Vilnius (LT); Giedrius Zolubas, Vilnius (LT)

(73) Assignee: Droplet Genomics UAB, Vilnius (LT)

(21) Appl. No.: 18/702,108

(22) PCT Filed: Oct. 24, 2022
(86) PCT No.: PCT/EP2022/079543

§ 371 Date: Apr. 17, 2024

(51) Int. Cl. B01J 13/20; B01J 13/04; B01J 13/14

(52) U.S. Cl. CPC B01J 13/206; B01J 13/04; B01J 13/14

(57) ABSTRACT

Provided in part are microparticle-containing microspheroids, processes for preparation, and methods for detecting microparticle optical patterns in the microspheroids.

### RELATED APPLICATION

This patent application claims the benefit of U.S. provisional application No. 63/273,445, filed on Oct. 29, 2021, entitled MICROSPHEROIDS CONTAINING DISTINGUISHABLE MICROPARTICLE PATTERNS, naming Giedrius ZOLUBAS et al. as inventors, and designated by attorney docket no. DRP-1002-PV. The entire content of the foregoing patent application is incorporated herein by reference for all purposes, including all text, tables and drawings.

### FIELD

The technology relates in part to microparticle-containing microspheroids, processes for preparation, and methods for differentiating microspheroids in a population by optically detecting distinguishable microparticle patterns in the microspheroids.

### SUMMARY

Analysis of microfluidic compartments in a population generally is limited to single-time snapshots without tracking individual compartments. The inability to distinguish individual microfluidic compartments from one another makes it impossible to track individual microfluidic compartments in a population overtime.

As a solution to this problem, provided herein are a plurality of microfluidic compartments in a population, referred to as microspheroids, having optically distinct micropatterns, and the manufacture and use of such microspheroids. Provision of microspheroids having distinguishable microparticle patterns permits optical tracking of large numbers of individual microfluidic compartments in a population. In certain aspects, provided is a population of microspheroid compartments in which a collection of microparticles is embedded in a sub-region of the microspheroids that form a random pattern unique to each of the microspheroids. The unique pattern can be detected and used to identify an individual microspheroid in the population of microspheroids, and track individual microspheroids at different time points and/or for different experimental conditions.

Provided in certain aspects are processes for preparing a plurality of hardened microparticle-localized microspheroids, the processes including: (a) providing a population of microparticle-containing microspheroids in which at least a subpopulation of the microspheroids contain a plurality of microparticles smaller than the microspheroids; (b) exposing, after part (a), the population of microparticle-containing microspheroids to sedimentation conditions that localize the microparticles to a microspheroid sub-region, thereby generating a plurality of microparticle-localized microspheroids containing the particles in a sub-region of each microspheroid; and (c) exposing the plurality of microparticle-localized microspheroids to hardening conditions, thereby generating a plurality of hardened microparticle-localized microspheroids.

Also provided in certain aspects is a plurality of hardened microparticle-localized microspheroids, obtainable by a process described herein.

Provided also in certain aspects are methods for differentiating a microspheroid from other microspheroids in a population of microspheroids, the methods including: (i) capturing an image of the microparticles in the sub-region of an individual microspheroid for multiple microspheroids in a plurality of hardened microparticle-localized microspheroids described herein, thereby capturing multiple microparticle images; (ii) differentiating one microparticle image from other microparticle images within the multiple microparticle images, thereby identifying a differentiated microparticle image; and (iii) differentiating, after (ii), an individual microspheroid from other microspheroids in the population of microparticle-containing microspheroids according to the differentiated microparticle image.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIGS. 1A-1D illustrate problems associated with the inability to track individual microspheroids in a population, and advantages afforded by optical pattern tracking.

FIGS. 2A-2D illustrate sedimentation of microparticles within a microspheroid and microparticle pattern properties in a sub-region of the microspheroid.

FIGS. 3A-3C illustrate microparticle pattern imaging in microspheroids (referred to as “droplet imaging” in this particular example), and examples of segmentation and rotational convolution approaches to pattern recognition.

FIG. 4 illustrates a non-limiting application of the technology described herein.

FIG. 5 illustrates another non-limiting application of the technology described herein.

FIGS. 6A-6B illustrate use of a magnetic field to align microspheroids for consistent imaging.

FIGS. 7A-7C illustrate microspheroid segmentation and optical encoding processes.

### DETAILED DESCRIPTION

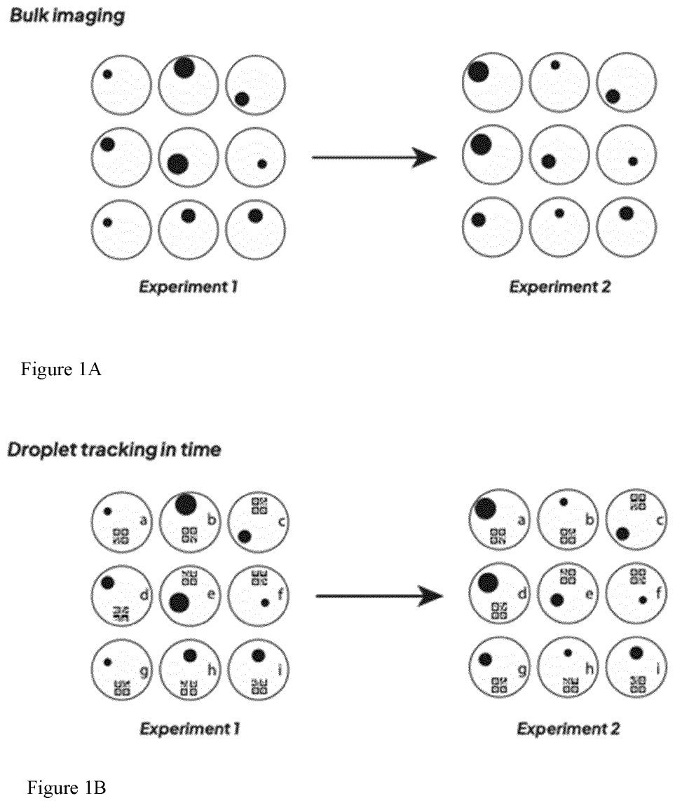

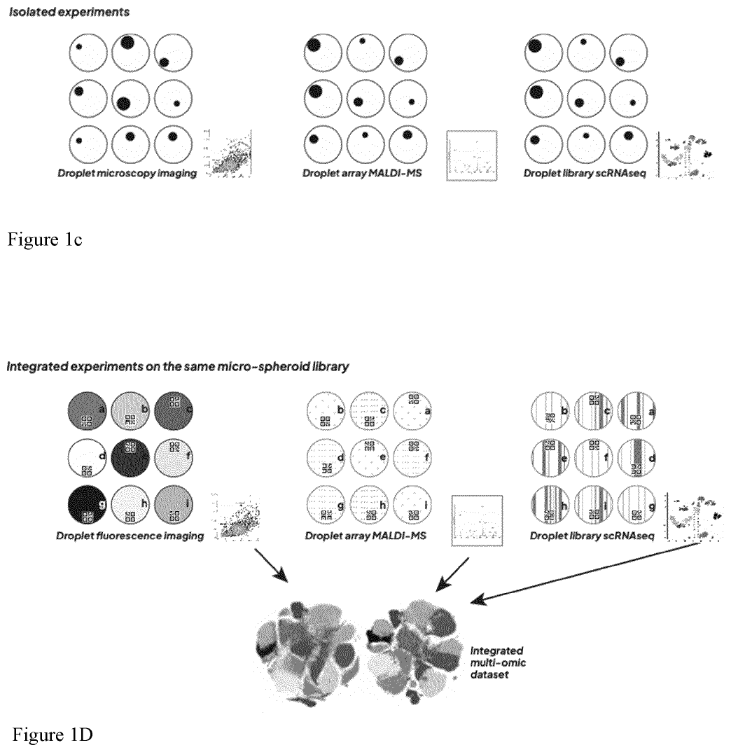

Analysis of microfluidic compartments in a population often is limited to single-time snapshots without tracking individual compartments. The inability to distinguish individual microfluidic compartments from one another makes it difficult or impossible to track individual microfluidic compartments in a population over time. This inability to distinguish individual microfluidic compartments is illustrated in FIG. 1A.

FIG. 1A illustrates a population of microspheroids having no distinguishable microparticle patterns. In these microspheroids, the black circle represents a detectable feature, such as cell size (e.g., where the black circle is a cell) or signal magnitude (e.g., where the size of the black circle represents signal magnitude). The detectable feature can be detected for each of the microspheroids in the population at one time point (e.g., “Experiment 1” on the left) and another time point (“Experiment 2” on the right). The inability to associate the detectable feature with an individual microsphere results in the inability to determine whether there is a larger or smaller cell or signal in Experiment 2 relative to Experiment 1 for an individual microsphere.

As a solution to this problem, provided herein are a plurality of microfluidic compartments in a population, referred to as microspheroids, having optically distinct microparticle patterns. Provision of microspheroids having distinguishable microparticle patterns permits optical tracking of large numbers of individual microfluidic compartments in a population. In certain aspects, provided is a population of microspheroid compartments in which a collection of microparticles is embedded in a sub-region of the microspheroids that form a random pattern unique to each of the microspheroids. The unique pattern can be detected and used to identify an individual microspheroid in the population of microspheroids, and facilitates tracking of individual microspheroids over time and/or for different experimental conditions.

FIG. 1B illustrates a population of microspheroids having distinguishable microparticle patterns. The patterns are, for illustration purposes only, depicted by rectangular codes in each of the microspheroids. The ability to define individual microspheroids by a unique microparticle pattern in each microspheroid permits tracking of each individual microspheroid, illustrated as individual microspheroids a-i in FIG. 1B. Distinguishing individual microspheroids in the population by distinguishable microparticle patterns permits determination of whether a microspheroid is associated with a larger or smaller cell or signal in Experiment 2 relative to Experiment 1. Distinguishing (i.e., differentiating) individual microspheroids in the population of microspheroids is accomplished by distinguishing each unique microparticle pattern associated with each microspheroid.

FIG. 1C and FIG. 1D are counterparts to FIG. 1A and FIG. 1B, respectively, and show that tracking of individual microspheroids in a population permits association of multiple characterizations of a particular microspheroid and subsequent provision of integrated datasets. In FIG. 1C, non-distinguishable microspheroids are subjected to different detection events, including optical imaging on the left, MALDI mass spectrometry in the middle and nucleic acid sequencing on the right. Without the ability to distinguish individual microspheroids from one another, it is impossible to associate each detection event with a particular microspheroid and integrate the three detection events associated with each microspheroid. In FIG. 1D, however, the ability to differentiate each microspheroid a-i for each of the detection events, by identification of the unique microparticle pattern associated with each microspheroid, permits integration of the three specific detection events with each particular microspheroid, and the provision of an integrated dataset shown in at the bottom of FIG. 1D.

### Microparticle-Containing Microspheroids and Methods of Manufacture

Provided herein are processes for preparing a plurality of hardened microparticle-localized microspheroids. Such processes can include: (a) providing a population of microparticle-containing microspheroids in which at least a subpopulation of the microspheroids contain a plurality of microparticles smaller than the microspheroids; (b) exposing, after part (a), the population of microparticle-containing microspheroids to sedimentation conditions that localize the microparticles to a microspheroid sub-region, thereby generating a plurality of microparticle-localized microspheroids containing the particles in a sub-region of each microspheroid; and (c) exposing the plurality of microparticle-localized microspheroids to hardening conditions, thereby generating a plurality of hardened microparticle-localized microspheroids. Also, provided is a plurality of hardened microparticle-localized microspheroids, obtainable by a process described herein.

### Microspheroids

The surface of a microspheroid generally is defined as an ellipsoid of revolution or a rotational ellipsoid. A microspheroid surface (i) generally is a quadric surface defined by rotating an ellipse or circle about one of its principal axes, (ii) generally is an ellipsoid with two equal semi-diameters, and (iii) generally has circular symmetry. A microspheroid is a prolate (elongated) microspheroid when the ellipse is rotated about its major axis. A microspheroid is an oblate (flattened) microspheroid when the ellipse is rotated about its minor axis. A microspheroid is a sphere (i.e., a microsphere) when the generating ellipse is a circle. A microspheroid surface sometimes is deformable and/or elastic, and one microspheroid sometimes can adopt a prolate, oblate or spherical shape at a particular point in time. A microspheroid can be prolate, oblate or spherical depending on the environment in which it is disposed. In a non-limiting example, a microspheroid can be an oblate spheroid when disposed in a channel of a fluidic device, where the channel is defined by a cross-sectional diameter less than the diameter of a spherical form of the microspheroid. In another non-limiting example, a microspheroid can be a sphere when disposed in a container having walls spaced by a distance greater than the diameter of a spherical form of the microspheroid and disposed in a volume that separates the microspheroid from other components. A microspheroid surface is not elastic and not deformable in certain implementations.

A microspheroid generally is defined by a diameter of about 1 micrometer to about 1,000 micrometers when in spherical form or when converted to a virtual sphere. In certain implementations, microspheroids (i.e., before hardening or after hardening) are defined by a diameter of about 30 micrometers to about 100 micrometers when in spherical form or when converted to a virtual sphere.

Any suitable type of microspheroid fluidic compartment can be utilized as an input component to prepare hardened microparticle-localized microspheroids. Non-limiting examples of microspheroids utilized to prepare microparticle-localized microspheroids include droplets, beads (e.g., hydrogel beads), vesicles and capsules. Microspheroids utilized to prepare microparticle-localized microspheroids can include one or more polymer components, and sometimes are referred to as polymeric microspheroids. Microspheroids can include one or more hydrogel polymers, non-limiting examples of which include agarose and gelatin. In certain implementations, two-component microspheroids are utilized to prepare hardened microparticle-localized microspheroids, which can include an interior comprising a first component and an exterior comprising a second component. The first component sometimes includes a polysaccharide (e.g., dextran). In certain implementations, the second component includes a cross-linking polymer, a non-limiting example of which includes a modified polyethylene glycol polymer suitable for cross-linking. In certain implementations, the first component includes a polysaccharide (e.g., dextran) and the second component includes a cross-linking polymer (e.g., modified polyethylene glycol polymer).

### Microparticles

Any suitable type of microparticle can be utilized to prepare hardened microparticle-localized microspheroids. Microparticles generally are smaller than each microspheroid in which they are contained. In certain implementations, microparticles generally are spheroidal in shape and have a diameter of about 1 micrometer to about 5 micrometers when in spherical form or converted to a virtual spherical form. Microparticles sometimes include one or more polymer components, sometimes include one or more metal components, and sometimes are beads. In certain implementations, microparticles are magnetic and can include, contain or consist of a magnetic component (e.g., a metal component that includes or consists of iron).

In certain implementations, a microparticle includes one or more detectable agents. In a population of microparticles, (i) sometimes microparticles are associated with a single detectable agent and (ii) sometimes a subpopulation of microparticles is associated with one detectable agent and another subpopulation of microparticles is associated with a different detectable agent. A detectable agent sometimes is linked to a microparticle, sometimes is located at a microparticle surface and/or sometimes is contained within a microparticle. Non-limiting examples of detectable agents include fluorescent labels such as organic fluorophores, lanthanide fluorophores (chelated lanthanides; dipicolinate-based Terbium (Ill) chelators), transition metal-ligand complex fluorophores (e.g., complexes of Ruthenium, Rhenium or Osmium); quantum dot fluorophores, isothiocyanate fluorophore derivatives (e.g., FITC, TRITC), succinimidyl ester fluorophores (e.g., NHS-fluorescein), maleimide-activated fluorophores (e.g., fluorescein-5-maleimide), and amidite fluorophores (e.g., 6-FAM phosphoramidite); radioactive isotopes (e.g., 1-125, 1-131, S-35, P-31, P-32, C-14, H-3, Be-7, Mg-28, Co-57, Zn-65, Cu-67, Ge-68, Sr-82, Rb-83, Tc-95m, Tc-96, Pd-103, Cd-109, and Xe-127); light scattering or light diffracting labels (e.g., light scattering gold nanorods, resonance light scattering particles); an enzymic or protein label (e.g., green fluorescence protein (GFP), peroxidase); or other chromogenic label or dye (e.g., cyanine). Non-limiting examples of organic fluorophores include xanthene derivatives (e.g., fluorescein, rhodamine, Oregon green, eosin, Texas red); cyanine derivatives (e.g., cyanine, indocarbocyanine, oxacarbocyanine, thiacarbocyanine, merocyanine); naphthalene derivatives (dansyl, prodan derivatives); coumarin derivatives; oxadiazole derivatives (e.g., pyridyloxazole, nitrobenzoxadiazole, benzoxadiazole); pyrene derivatives (e.g., cascade blue); oxazine derivatives (e.g., Nile red, Nile blue, cresyl violet, oxazine 170); acridine derivatives (e.g., proflavin, acridine orange, acridine yellow); arylmethine derivatives (e.g., auramine, crystal violet, malachite green); and tetrapyrrole derivatives (e.g., porphin, phtalocyanine, bilirubin).

In certain implementations, microparticle-containing microspheroids on average (e.g., mean, median) contain about 5 microparticles to about 100 microparticles. Microparticles can be incorporated into microspheroids by any suitable process. Microparticles sometimes are combined with components that form microspheroids prior to or during formation of the microspheroids, and are incorporated within the microspheroids under conditions that form the microspheroids. In certain implementations, a process includes (i) contacting one or more microspheroid-forming components with a plurality of microparticles, and (ii) forming microspheroids under formation conditions in which multiple microparticles are incorporated within formed microspheroids in a population of the formed microspheroids, thereby providing a population of microparticle-containing microspheroids. Sometimes the microspheroid-forming components are droplet-forming components, and the microparticles are incorporated into formed droplets upon formation of the droplets. Suitable processes for forming droplets and other microspheroids are known, and sometimes droplets are formed from droplet-forming components by utilizing a microfluidic chip. In certain implementations, microparticles may be incorporated into microspheroids after microspheroids that do not contain microparticles have been formed.

### Microparticle Sedimentation

Any suitable process can be utilized to sediment microparticles incorporated in the microspheroids. In certain implementations, the microparticles are of a first density and the microspheroids are of a second density less than the first density, and the sedimentation conditions include sedimenting the microparticles by gravity for a period of time to the sub-region of the microparticle-containing microspheroids. The period of time sometimes is about 10 minutes to about 2 hours. In certain implementations, sedimentation conditions include exposure of the microspheroids to a force consisting of gravity. Sedimentation conditions sometimes include exposure of the microspheroids to centrifugation.

In certain implementations, the microparticles are magnetic and the sedimentation conditions include applying a magnetic field to the microparticle-containing microspheroids that sediments the microparticles to the sub-region of the microspheroids. Any suitable magnetic field can be applied to the microparticles, and sometimes a magnet is placed in proximity to the microparticles in the microspheroids.

After sedimentation, for a majority of the microparticle-localized microspheroids, a majority of microparticles, or all microparticles, are disposed within the microspheroid sub-region, in certain implementations. In certain implementations, for a majority of the microparticle-localized microspheroids, a majority of microparticles, or all microparticles, are disposed in a microparticle monolayer. In the foregoing implementations, the majority of microparticle-localized microspheroids sometimes is more than 50%, about 60% or more, about 70% or more, about 80% or more, about 85% or more, about 90% or more, about 95% or more, or about 99% or more of total microparticle-localized microspheroids. In the majority of, or all of, the microparticle-localized microspheroids, sometimes about 80% or more, about 85% or more, about 90% or more, about 95% or more, about 99% or more, or about 100% of total microparticles in the microparticle-localized microspheroids are disposed in a microspheroid sub-region, or in a monolayer, or in a microspheroid sub-region and a monolayer.

A microspheroid sub-region is a microspheroid cap in certain implementations. A microspheroid cap generally is a portion of a microspheroid defined by a plane (i.e., a cutoff plane), and typically is a microspheroid segment bounded by a single plane. A microspheroid cap can be defined by (i) a radius a of the base of the cap, which is less than the radius r of the microspheroid in spherical form or virtual spherical form, (ii) a height h of the cap, and (iii) a polar angle theta between the rays from the center of a virtual sphere containing the microspheroid to the apex of the cap (the pole) and the edge of the disk forming the base of the cap. In certain instances, microspheroid radius r is an average (e.g., mean, median) radius of microspheroids in a population, and: (i) the cap radius a is less than ⅔ of microspheroid radius r, or (ii) the cap height h is less than half of the microspheroid radius r, or (iii) the cap radius a is less than ⅔ of microspheroid radius rand the cap height h is less than half of the microspheroid radius r.

In certain implementations, a microspheroid sub-region to which microparticles are sedimented is of a surface area of about 25% or less of the total microspheroid surface area. In certain instances, the microspheroids are of an average diameter of about 60 micrometers and the microspheroid sub-region is of a surface area of about 3,000 square micrometers.

In certain implementations, a microspheroid sub-region to which microparticles are sedimented is of a volume of about 15% or less of the total microspheroid volume, and in certain instances is of a volume of about 15,000 cubic micrometers or about 15 picoliters. In certain implementations, the microspheroid sub-region is of a volume of about 1% or less of the total microspheroid volume. In certain instances, the microspheroid sub-region is of a volume of about 200 cubic micrometers or about 0.2 picoliters, e.g., for relatively small microparticles.

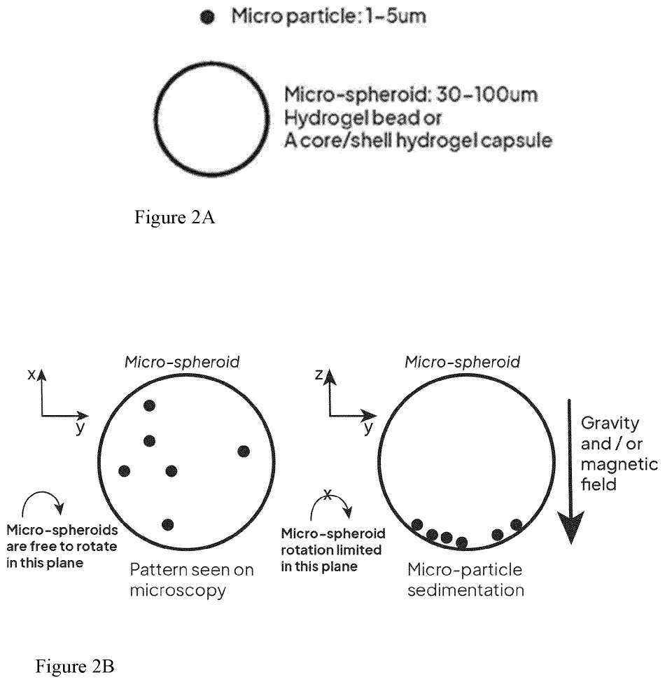

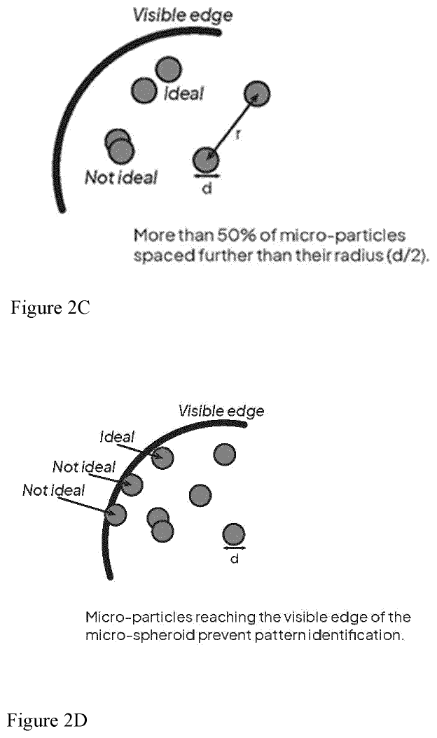

Non-limiting examples of microparticles and microspheroids are depicted in FIG. 2A and FIG. 2B. FIG. 2A illustrates particular size ranges of microparticles and microspheroids. FIG. 2B illustrates on the left a “bottom view” visualized in two dimensions (i.e., x and y dimensions) of a non-limiting example of a microparticle-localized microspheroid. FIG. 2B also illustrates on the right a “bottom view” visualized in two dimensions (i.e., x and z dimensions) of the same non-limiting example of the microparticle-localized microspheroid. FIG. 2B illustrates non-limiting examples of a force (i.e., gravity, magnetic field) and the direction of the force (i.e., parallel to the z-axis) utilized to sediment microparticles into the sub-region of the microspheroid.

### Microspheroid Hardening and Processing of Hardened Microspheroids

Any suitable process can be utilized to harden microspheroids after microparticles have been sedimented to a microspheroid sub-region. Hardening the microspheroids can fix or substantially fix the positions of microparticles in the sub-region of the microspheroids to which they have been localized by sedimentation, thereby facilitating consistent identification of a distinguishable microparticle pattern in individual microspheroids.

In certain implementations, the microspheroids include a thermally responsive hydrogel polymer, and the hardening conditions include cooling the microparticle-localized microspheroids to a temperature below a sol-to-gel transition temperature. A hydrogel polymer sometimes is agarose or gelatin either in natural or chemically modified form. The sol-to-gel transition temperature is about 25° C. for gelatin and is about 40° C. for agarose.

In certain implementations, the microspheroids include a cross-linking polymer, and the hardening conditions include exposing the microparticle-localized microspheroids to cross-linking conditions. Cross-linking conditions depend on the polymer(s) incorporated in the microspheroids and appropriate hardening conditions can be selected. Non-limiting examples of cross-linking conditions include (i) exposure to light, such as 365 nm or 405 nm wavelength illumination at 0.1 mW/cm² to 100 mW/cm² power, in the case of light-activated polymerization or cross-linking; (ii) exposure to free-radical generating chemical initiators, such as APS and TEMED, in the case of chemically-induced free-radical polymerization or cross-linking; and (iii) exposure to elevated temperatures for thermally-activated free-radical initiators, such as persulphate salts, in the case of thermally induced free-radical polymerization or cross-linking.

Hardened microparticle-localized microspheroids include, without limitation, droplets, beads (e.g., hydrogel beads), vesicles and capsules, including two-component microspheroids described herein (e.g., a microspheroid including an interior that contains a first component and an exterior that contains a second component). In certain implementations, hardened microparticle-localized microspheroids are isolated and/or separated from other components. In certain implementations, microspheroids are separated by straining through a mesh (e.g., a filter) with a size selected to retain the microspheroids and permit other smaller components to flow through the mesh and separate from the microspheroids. In certain implementations, microspheroids are sedimented from a suspension or emulsion by centrifugation. In centrifugation implementations, a continuous phase that is present can be removed or replaced. Hardened microparticle-localized microspheroids often are maintained in a composition comprising a fluid, and often are not dried.

### Analytes, Reagents and Biological Entities

In certain implementations, a hardened microparticle-localized microspheroid includes one or more components chosen independently from an analyte, a reagent and a biological entity. Any suitable analyte can be incorporated in a microspheroid, non-limiting examples of which include an inorganic analyte (e.g., metal, salt) and organic analyte (e.g., small molecule entity (e.g., small molecule drug) and organic biological entity (addressed herein)). Any suitable reagent can be incorporated in a microspheroid, non-limiting examples of which include a buffer, nucleotide, detectable agent (described herein), amino acid, enzyme (e.g., ligase, polymerase, transposase), antibody, cross-linking agent and the like. Any suitable biological entity can be incorporated in a microspheroid, non-limiting examples of which include a cell (e.g., prokaryotic cell (e.g., bacterium), eukaryotic cell (e.g., mammalian cell)), nucleic acid, peptide, polypeptide, saccharide, polysaccharide, fatty acid, lipid, and cholesterol and like.

An analyte, a reagent and/or a biological entity (i.e., collectively an application component) can be incorporated into microspheroids using any suitable process. An application component sometimes is combined with components that form microspheroids prior to or during formation of the microspheroids, and are incorporated within the microspheroids under conditions that form the microspheroids. In certain implementations, a process includes (i) contacting one or more microspheroid-forming components with an application component, and (ii) forming microspheroids under formation conditions in which the application component is incorporated within formed microspheroids in a population of the formed microspheroids, thereby providing a population of application component-containing microspheroids. Sometimes microspheroid-forming components are droplet-forming components, and the application component is incorporated into formed droplets upon formation of the droplets. Suitable processes for forming droplets and other microspheroids are known, and sometimes droplets are formed from droplet-forming components by utilizing a microfluidic chip. In certain implementations, an application component may be incorporated into microspheroids after microspheroids that do not contain the application component have been formed (e.g., incorporation by diffusing one or more application components into formed microspheres).

### Differentiation of Microparticle-Containing Microspheroids in a Population

In certain implementations, provided are methods for differentiating a microspheroid from other microspheroids in a population of microspheroids, which methods include: (i) capturing an image of the microparticles in the sub-region of an individual microspheroid for multiple microspheroids in the plurality of hardened microparticle-localized microspheroids described herein, thereby capturing multiple microparticle images; (ii) differentiating one microparticle image from other microparticle image within the multiple microparticle images, thereby identifying a differentiated microparticle image; and (iii) differentiating, after (ii), an individual microspheroid from other microspheroids in the population of microparticle-containing microspheroids according to the differentiated microparticle image. Each microparticle image captured in part (i) sometimes substantially is a two-dimensional image. Each microparticle image captured in part (i) sometimes is captured by optical detection, such as by microscopy (e.g., bright field microscopy), and/or by fluorescence detection (e.g., in instances where the microparticles are excited and emit a fluorescence signal), and/or by a spectroscopic detection (e.g., Raman spectroscopy or mass spectrometry), for example.

In certain implementations, the microparticle images captured in part (i) are for microspheroid sub-regions that are consistently oriented relative to a detector utilized to capture a microparticle image. In certain instances, the microparticle images captured in part (i) are identified in microspheres that can rotate in a first plane and are rotationally constrained in a second plane. In such instances, microparticle patterns generally are identified in the first plane. Microspheroids can be oriented and rotationally constrained by any suitable force, including without limitation, gravity (e.g., where microparticles are of a density greater than the microspheroid density) and/or magnetic field (e.g., where microparticles are magnetic). The first plane of a microspheroid can be oriented towards a detector, and/or opposite to the detector, utilized to capture a microparticle image. Without being limited by theory, consistent orientation of microspheroids in a first plane relative to a detector, and when applicable consistent rotational constraint of microspheroids in a second plane, permits accurate identification of microparticle patterns.

In certain instances, microparticles are magnetic, and the sub-region orientation of each of the microspheroids is consistently oriented by application of a magnetic field (e.g., generated by a magnet). In certain implementations, the microparticle images captured in part (i) are captured by a detector element, and the sub-region is oriented towards the detector element. In certain instances, the microparticle image is captured by a microscope that includes a microscope objective, and the sub-region is oriented opposite to (i.e., facing) the microscope objective. FIG. 2B illustrates on the left microparticle image capture of a sub-region visualized in two dimensions (i.e., the x and y dimensions), where the sub-region visualized in the x and y plane is opposite the microscope objective (i.e., the microscope objective visualizes the sub-region in the x and y plane, which is referred to as the first plane in the paragraph directly above). The microspheroid illustrated on the left is oriented with the sub-region visualized in the x and y dimensions and is free to rotate in the x and y plane (i.e., the first plane). FIG. 2B illustrates on the right the same microspheroid as depicted on the left, but illustrated in the x and z plane with the sub-region located in the bottom of the microspheroid. As depicted on the right in FIG. 2B, the microspheroid sub-region is oriented opposite the microscope objective positioned in the z-dimension, pointing upwards and visualizing the bottom of the microspheroid in the z-dimension. The microspheroid is rotationally constrained in the x and z plane (i.e., the second plane) as depicted in FIG. 2B.

In certain implementations, the microparticle images differentiated in part (ii) are for microspheroids in which 50% or more of the microparticles are not overlapping. In certain instances, each of the microparticles is defined by a microparticle radius, and the microparticle images differentiated in part (ii) are for microspheroids in which 50% or more of the microparticles are spaced a distance greater than the microparticle radius from other microparticles. FIG. 2C illustrates a non-limiting example of a microparticle-containing microspheroid in which more than 50% of the microparticles are separated by a distance r greater than the microparticle radius (d/2) (e.g., microspheroids in which about 60% or more, 70% or more, 80% or more, 90% or more or 95% or more of the microparticles are separated by a distance r greater than the microparticle radius (d/2)). In certain implementations, each microspheroid includes a visible microspheroid edge defined by optical detection, and the microparticle images differentiated in part (ii) are for microspheroids containing no microparticles contacting the visible microspheroid edge, and/or for microspheroids in which more than 50% of the microparticles are not contacting the visible microspheroid edge (e.g., microspheroids in which about 60% or more, 70% or more, 80% or more, 90% or more or 95% or more of the microparticles do not contact the visible microspheroid edge). FIG. 2D illustrates a non-limiting example of a microparticle-containing microspheroid in which a subset of microparticles contact the visible microspheroid edge (i.e., designated as “not ideal”) and a subset of microparticles do not contact the visible edge (i.e., designated as “ideal”). A visible microspheroid edge can be identified and/or visualized using any suitable type of optical detection, a non-limiting example of which includes detection by a light microscope (e.g., bright-field microscope).

In certain implementations, a method for differentiating a microspheroid from other microspheroids includes: capturing microparticle images and detecting a detectable feature for microparticle-containing microspheroids in a population of microparticle-containing microspheroids at a first point in time; capturing microparticle images and detecting a detectable feature for microparticle-containing microspheroids in a population of microparticle-containing microspheroids at a second point in time; differentiating individual microparticle images each associated with an individual microspheroid from the microparticle images captured at the first point of time and the second point of time; and detecting the presence or absence of a change in the detectable feature between the first point in the time and the second point in time for individual microspheroids according to the individual microparticle images. In certain instances, detecting the detectable feature includes detection by one or more of microscopy, nucleic acid sequencing and spectrometry. The spectrometry in certain instances is mass spectrometry, or Raman spectrometry, or mass spectrometry and Raman spectrometry.

A microparticle pattern can be associated with a particular microspheroid and differentiated from the microparticle patters of other microspheroids in a process that includes capturing an image of microparticles in a microspheroid or microspheroids, thereby generating a captured microparticle image. A microparticle image can be captured in any suitable manner and can be captured by a detector. A microparticle detectable feature generally is detected in microspheroids as part of capturing the microparticle images in part (i). In certain instances, the detectable feature is light image captured by a microscope. In certain implementations, the detectable feature is fluorescence emitted by a fluorophore associated with microparticles, which can be detected by a photon detector. In certain instances, microparticle images are captured utilizing a microscopic image of the microparticles and a fluorescence image of the microparticles also is captured. Non-limiting examples of detectors include a camera (e.g., a camera integrated with a microscope), a photon detector or other spectrophotometric detector (e.g., suitable for microparticle imaging by fluorescence detection and/or Raman spectroscopy). A camera sometimes is a digital imaging camera, and can include a charge-coupled device (CCD) sensor in certain implementations. A captured microparticle image can contain an image of microparticles within one or more microspheroids. A microparticle image, and often multiple microparticle images, can be stored in silico and sometimes are stored in silico in a library of microparticle images used for microspheroid identification purposes.

In certain instances, differentiating an image includes segmenting in silico a captured image, thereby generating a segmented microparticle image (i.e., a sub-image). As microspheroids are round, a Hough circle detection process sometimes is utilized for segmentation. A segmented image can include microparticles within one microspheroid or a portion of a microspheroid.

In certain instances, differentiating a microparticle image can include cropping a captured microparticle image and/or a segmented microparticle image, thereby generating a cropped microparticle image (i.e., a sub-image). A cropped microparticle image can include microparticles within one microspheroid or a portion of a microspheroid. A cropped microparticle image may or may not include microparticles in one or more other microspheroids present in the captured microparticle image.

A captured microparticle image, a segmented microparticle image, and/or a cropped microparticle image can be utilized in a differentiation process described herein, and are referred to collectively as a “microparticle image” when addressing differentiation processes. In certain implementations, a differentiation process can include optically encoding a microparticle image associated with a microspheroid, thereby generating an optically encoded microparticle image. A microparticle image can be optically encoded in any suitable manner. In a differentiation process, a test microparticle image for one microspheroid can be compared, sometimes in an iterative image-by-image process, to comparison microparticle images of microspheroids within a library of comparison microparticle images. Each comparison can involve rotating the test microparticle image in increments (e.g., one-degree increments) and generating one or more scores based on each comparison image (e.g., by image convolution). A score can be generated for each of the comparison images and/or for each increment. A particular microparticle image, depicting a particular microparticle pattern, can be associated with a particular microspheroid and differentiated form microparticle images of other microspheroids based on the scores.

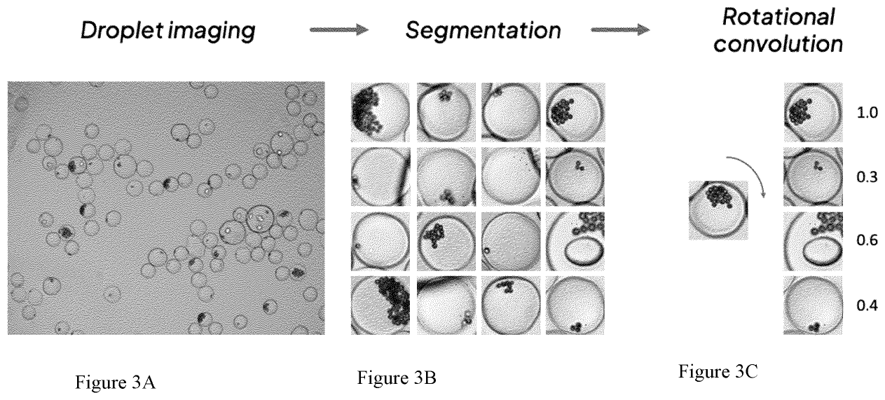

A non-limiting example of an optical encoding process is illustrated in FIGS. 3A-3C. FIG. 3A illustrates particle images from multiple microspheroids, which in the instance shown are droplets. FIG. 3B illustrates a segmentation process in which the captured microparticle image is segmented into sub-images each comprising a microparticle image for predominantly one microspheroid. FIG. 3C illustrates an optical encoding process in which one of the segmented sub-images (i.e., a test sub-image) is rotated and compared to other comparison sub-images. A score is generated for each of the comparison sub-images (i.e., a score of 0.3, 0.4, 0.6 and 1.0). The microparticle image, i.e., the test sub-image, is associated with the comparison sub-image having the highest score (i.e., 1.0) and differentiated from the other microparticle images associated with other microspheroids. The microspheroid from which the associated microparticle image (i.e., microparticle pattern) having the highest score is captured (and optionally processed) thereby is differentiated from other microspheroids in the population.

As an alternative approach to optical encoding, or in combination with optical encoding, a differentiation process can include generating coordinates (e.g., x and y coordinates) for the microparticles within a microparticle image and generating a nearest neighbor graph containing the coordinates. In a nearest neighbor graph, the microparticles generally are the nodes and their distances from each other generally are graph edges. The microparticle graphs for each microparticle image can be stored in silico as encodings and graph alignment processes can be utilized to compare a graph for one microparticle image to a library of graphs corresponding to comparison microparticle images, thereby generating a graph comparison. A microparticle image (i.e., a microparticle pattern) can be associated with a particular microspheroid and differentiated form microparticle images of other microspheroids using graph comparison processes. Non-limiting examples of graph comparison processes are addressed in Chen et al., “Unsupervised Adversarial Graph Alignment with Graph Embedding” ArXiv abs/1907.00544 (2019) and at World Wide Web URL github.com/GemsLab/REGAL. A microspheroid from which the associated microparticle image (i.e., microparticle pattern) has been captured (and optionally processed) thereby is differentiated from other microspheroids in the population.

Each of the microparticle images captured generally define an individual microparticle pattern associated with a particular microspheroid. The microparticle pattern is defined by the locations of microparticles in the microspheroid sub-region containing the majority or all of the microparticles. In a microparticle-localized microspheroid population, there sometimes exists a microparticle pattern diversity of about 1,000 to about 100 million or more (e.g., about 1,000 to about 100 million, about 1,000 to about 10 million, about 1,000 to about 1 million; about 1,000 to about 500,000; about 1,000 to about 250,000; about 1,000 to about 100,000, about 1,000 to about 10,000, about 1,000 to about 5,000) microparticle patterns that can be differentiated from one another (i.e., unique microparticle patterns, individual microparticle patterns; different microparticle patterns).

### Kits

Provided in certain embodiments are kits. A kit may include any component and/or composition described herein (e.g., microparticles) useful for performing any of the methods described herein, in any suitable combination. A kit may include a microparticle component, a component for forming droplets (e.g., aqueous solution, oil, surfactant, fluidic device), a hardening component, a magnet, an optical device, an imaging device, a reagent, a buffer and/or other component suitable for carrying out a method described herein.

Components of a kit may be present in separate containers, and/or multiple components may be present in a single container. Suitable containers include a single tube (e.g., vial), one or more wells of a plate (e.g., a 96-well plate, a 384-well plate, and the like), and the like.

A kit may include instructions for performing one or more methods described herein and/or a description of one or more components described herein. For example, a kit may include instructions for using microparticles to manufacture hardened microsphereoids described herein, may include instructions for identifying a microparticle pattern in a microspheroid, and/or may include instructions for differentiating a microparticle pattern in a microspheroid from another microparticle pattern in another microspheroid. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit may include a written description of an internet location that provides such instructions and/or descriptions.

### Non-Limiting Applications of the Technology

FIG. 1D illustrates an application of the technology that permits experimental integration. A challenge for experimental integration is tracking individual compartments between different imaging, spectrometry or sequencing experiments. The application illustrated in FIG. 1D outlines an example of performing cytometry, then taking the microspheroids containing cells to the substrate of a MALDI-TOF for mass spectrometry and then placing the particles in barcoded 96 well plates for genomic or transcriptomic readout. This application facilitates linking of the genomic, proteomic and metabolomic information into a single multi-omic dataset.

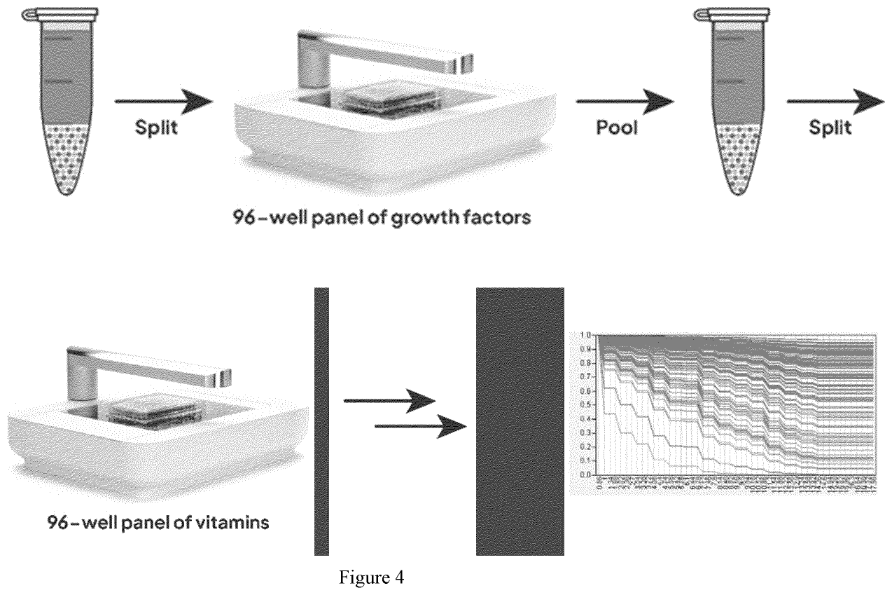

FIG. 4 illustrates a particular application of the technology that permits cell tracking between experiments. Compartmentalized cells are measured and tracked as they are translated between various conditions. The compartments generally are microspheroids, such as droplets. The cells sometimes are prokaryotic cells (e.g., bacterial cells) and sometimes are eukaryotic cells (e.g., mammalian cells). Collections of encapsulated cells are placed in 96-well plates with panels of inhibitors/growth conditions/transfection vectors. This application permits testing of sequential conditions for cell culture and/or cell line development. A collection of about 10,000 to about 100,000 microspheroids containing cells in wells can be analyzed in a split-pool fashion. This application permits relation of cell growth or fluorescence to the conditions in which cells are placed.

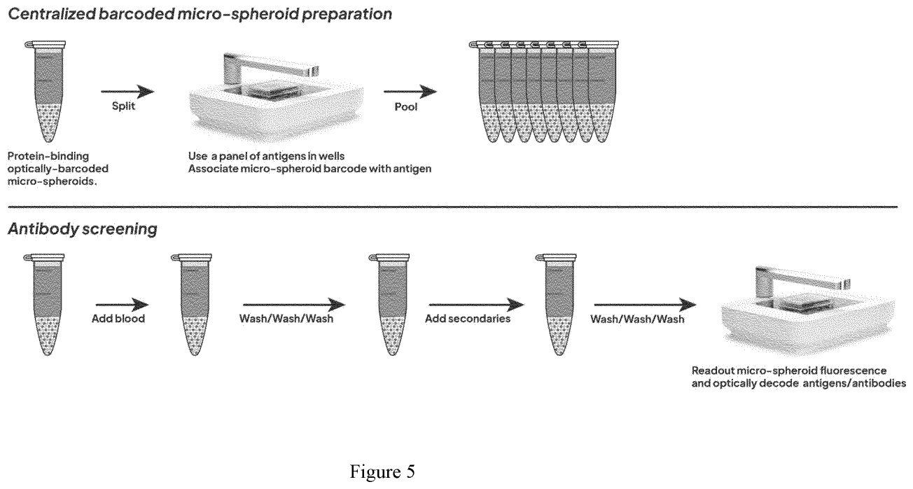

FIG. 5 illustrates a particular application of the technology that facilitates multiplex binding assays. Microspheroids carry a library of reagents that can be decoded after a reaction. One example of such an approach is using a library of antigen-carrying beads to perform a high-throughput binding assay against blood antibodies.

### Certain Implementations

Following are non-limiting implementations of the technology.

A1. A process for preparing a plurality of hardened microparticle-localized microspheroids, comprising:
(a) providing a population of microparticle-containing microspheroids in which at least a subpopulation of the microspheroids contain a plurality of microparticles smaller than the microspheroids;
(b) exposing, after part (a), the population of microparticle-containing microspheroids to sedimentation conditions that localize the microparticles to a microspheroid sub-region, thereby generating a plurality of microparticle-localized microspheroids containing the particles in a sub-region of each microspheroid; and
(c) exposing the plurality of microparticle-localized microspheroids to hardening conditions, thereby generating a plurality of hardened microparticle-localized microspheroids.
   A2. The process of embodiment A1, wherein the microspheroids in part (a) are droplets.
   A3. The process of embodiment A1, wherein the microspheroids in part (a) are hydrogel beads.
   A4. The process of embodiment A1, wherein the microspheroids in part (a) are two-component microspheroids.
   A5. The process of embodiment A4, wherein the microspheroids in part (a) comprise an interior comprising a first component and an exterior comprising a second component.
   A6. The process of embodiment A5, wherein the second component is a cross-linking polymer.
   A7. The process of embodiment A6, wherein the first component comprises dextran and the second component comprises a modified polyethylene glycol polymer.
   A8. The process of any one of embodiments A1-A7, wherein the hardened microparticle-localized microspheroids are about 30 micrometers to about 100 micrometers in diameter.
   A9. The process of any one of embodiments A1-A8, wherein the microparticles are about 1 micrometer to about 5 micrometers in diameter.
   A10. The process of any one of embodiments A1-A9, wherein the microparticles are magnetic.
   A11. The process of any one of embodiments A1-A10, wherein the microparticles comprises a detectable agent.
   A12. The process of embodiment A11, wherein the detectable agent comprises a fluorophore.
   A13. The process of any one of embodiments A1-A12, wherein the microparticles are of a first density and the microspheroids in part (a) are of a second density less than the first density.
   A14. The process of any one of embodiments A1-A13, wherein the microparticle-containing microspheroids in part (a) on average contain about 5 microparticles to about 100 microparticles.
   A15. The process of any one of embodiments A1-A14, wherein:
the microparticles are of a first density and the microspheroids are of a second density less than the first density; and
the sedimentation conditions in part (b) comprise sedimenting the microparticles by gravity for a period of time to the sub-region of the microparticle-containing microspheroids.
   A16. The process of embodiment A15, wherein the period of time is about 10 minutes to about 2 hours.
   A17. The process of embodiment A15 or A16, wherein the sedimentation conditions include exposure of the microspheroids to a force consisting of gravity.
   A18. The process of embodiment A15 or A16, wherein the sedimentation conditions comprise exposure of the microspheroids to centrifugation.
   A19. The process of any one of embodiments A1-A18, wherein:
the microparticles are magnetic; and
the sedimentation conditions in part (b) comprise applying a magnetic field to the microparticle-containing microspheroids that sediments the microparticles to the sub-region of the microspheroids.
   A20. The process of any one of embodiments A1-A19, wherein:
the microspheroids comprise a thermally responsive hydrogel polymer, and
the hardening conditions in part (c) comprise cooling the microparticle-localized microspheroids to a temperature below a sol-to-gel transition temperature.
   A21. The process of embodiment A20, wherein the hydrogel polymer is agarose or gelatin.
   A22. The process of any one of embodiments A1-A21, wherein:
the microspheroids comprise a cross-linking polymer; and
the hardening conditions in part (c) comprise exposing the microparticle-localized microspheroids to cross-linking conditions.
   A23. The process of any one of embodiments A1-A22, comprising isolating the hardened microparticle-localized microspheroids.
   A24. The process of embodiment A23, comprising separating the hardened microparticle-localized microspheroids from other components.
   A25. The process of any one of embodiments A1-A24, wherein the hardened microparticle-localized microspheroids comprise a biological entity.
   A26. The process of embodiment A25, wherein the biological entity is chosen from a cell, nucleic acid, peptide, polypeptide, saccharide, polysaccharide, fatty acid, lipid, and cholesterol.
   A27. The process of any one of embodiments A1-A26, wherein the hardened microparticle-localized microspheroids comprise a reagent.
   A28. The process of any one of embodiments A1-A27, wherein, for a majority of the hardened microparticle-localized microspheroids, the majority of microparticles in the hardened microparticle-localized microspheroids are disposed in the microspheroid sub-region.
   A29. The process of embodiment A28, wherein the majority of hardened microparticle-localized microspheroids is more than 50%.
   A30. The process of embodiment A28, wherein the majority of hardened microparticle-localized microspheroids is more than 60%.
   A31. The process of embodiment A28, wherein the majority of hardened microparticle-localized microspheroids is more than 70%.
   A32. The process of embodiment A28, wherein the majority of hardened microparticle-localized microspheroids is more than 80%.
   A33. The process of embodiment A28, wherein the majority of hardened microparticle-localized microspheroids is more than 90%.
   A34. The process of embodiment A28, wherein the majority of hardened microparticle-localized microspheroids is more than 95%.
   A35. The process of embodiment A28, wherein the majority of hardened microparticle-localized microspheroids is more than 99%.
   A36. The process of embodiment A28, wherein the majority of hardened microparticle-localized microspheroids is 100%.
   A37. The process of any one of embodiments A28-A36, wherein 80% or more of the microparticles in the hardened microparticle-localized microspheroids are disposed in the microspheroid sub-region.
   A38. The process of embodiment A37, wherein 85% or more of the microparticles in the hardened microparticle-localized microspheroids are disposed in the microspheroid sub-region.
   A39. The process of embodiment A37, wherein 90% or more of the microparticles in the hardened microparticle-localized microspheroids are disposed in the microspheroid sub-region.
   A40. The process of embodiment A37, wherein 95% or more of the microparticles in the hardened microparticle-localized microspheroids are disposed in the microspheroid sub-region.
   A41. The process of any one of embodiments A1-A40, wherein, for a majority of the hardened microparticle-localized microspheroids, the majority of microparticles are disposed in a monolayer in the microspheroid sub-region of the hardened microparticle-localized microspheroids.
   A42. The process of any one of embodiments A1-A41, wherein the microspheroid sub-region is a microspheroid cap.
   A43. The process of embodiment A42, wherein: the microparticle-localized microspheroids are defined by an average microspheroid radius r, and: (i) the microspheroid cap comprises a radius a less than ⅔ of the microspheroid radius r, or (ii) the microspheroid cap comprises a height h less than half of the microspheroid radius r, or (iii) the microspheroid cap radius a is less than ⅔ of the microspheroid radius rand the microspheroid cap height h is less than half of the microspheroid radius r.
   A44. The process of any one of embodiments A1-A43, wherein the microspheroid sub-region is of a surface area of about 25% or less of the total microspheroid surface area.
   A45. The process of any one of embodiments A1-A43, wherein the microspheroid sub-region is of a volume of about 15% or less of the total microspheroid volume.
   A46. The process of any one of embodiments A1-A43, wherein the microspheroid sub-region is of a volume of about 1% or less of the total microspheroid volume.
   A47. The process of any one of embodiments A1-A46, comprising, prior to part (a):
contacting one or more microspheroid-forming components with a plurality of microparticles; and
forming microspheroids under formation conditions in which multiple microparticles are incorporated within microspheroids in a population of formed microspheroids, thereby providing the population of microparticle-containing microspheroids in part (a).
   A48. The process of embodiment A47, wherein the microspheroid-forming components are droplet-forming components and the microparticles are incorporated into formed droplets.
   B1. A composition, comprising a plurality of hardened microparticle-localized microspheroids, obtainable by a process according to any one of embodiments A1-A48.
   B2. A composition, comprising a plurality of microparticle-localized microspheroids, wherein each of the microspheroids comprises a plurality of microparticles within a microspheroid sub-region.
   B3. The composition of embodiment B2, wherein the microspheroids are hardened microspheroids.
   B4. The composition of any one of embodiments B1-B3, wherein for a majority of, or all of, the microparticle-localized microspheroids, a majority of, or all of, the microparticles in each of the microparticle-localized microspheroids is/are disposed within the microspheroid sub-region.
   B5. The composition of any one of embodiments B1-B4, wherein for a majority of, or all of, the microparticle-localized microspheroids, a majority of, or all of, the microparticles in each of the microparticle-localized microspheroids is/are disposed in a microparticle monolayer.
   B6. The composition of any one of embodiments B1-B5, wherein:
the microparticles in the microparticle-localized microspheroids are defined by an average microparticle radius (d/2); and
for a majority of, or all of, the microparticle-localized microspheroids, a majority of, or all of, the microparticles in each of the microparticle-localized microspheroids is/are separated by a distance r greater than the microparticle radius (d/2).
   B7. The composition of any one of embodiments B1-B6, wherein:
each microspheroid includes a visible microspheroid edge defined by optical detection; and
for a majority of, or all of, the microparticle-localized microspheroids, a majority of, or all of, the microparticles in each of the microparticle-localized microspheroids do not contact the visible microspheroid edge.
   B8. The composition of any one of embodiments B4-B7, wherein the majority of microparticle-localized microspheroids is more than 50% of total microparticle-localized microspheroids in the composition.
   B9. The composition of any one of embodiments B4-B7, wherein the majority of microparticle-localized microspheroids is about 60% or more of total microparticle-localized microspheroids in the composition.
   B10. The composition of any one of embodiments B4-B7, wherein the majority of microparticle-localized microspheroids is about 70% or more of total microparticle-localized microspheroids in the composition.
   B111. The composition of any one of embodiments B4-B7, wherein the majority of microparticle-localized microspheroids is about 80% or more of total microparticle-localized microspheroids in the composition.
   B12. The composition of any one of embodiments B4-B7, wherein the majority of microparticle-localized microspheroids is about 85% or more of total microparticle-localized microspheroids in the composition.
   B13. The composition of any one of embodiments B4-B7, wherein the majority of microparticle-localized microspheroids is about 90% or more of total microparticle-localized microspheroids in the composition.
   B14. The composition of any one of embodiments B4-B7, wherein the majority of microparticle-localized microspheroids is about 95% or more of total microparticle-localized microspheroids in the composition.
   B15. The composition of any one of embodiments B4-B7, wherein the majority of microparticle-localized microspheroids is about 99% or more of total microparticle-localized microspheroids in the composition.
   B16. The composition of any one of embodiments B4-B15, wherein the majority of the microparticles in each of the microparticle-localized microspheroids is about 75% or more of total microparticles in each of the microparticle-localized microspheroids.
   B16. The composition of any one of embodiments B4-B15, wherein the majority of the microparticles in each of the microparticle-localized microspheroids is about 80% or more of total microparticles in each of the microparticle-localized microspheroids.
   B17. The composition of any one of embodiments B4-B15, wherein the majority of the microparticles in each of the microparticle-localized microspheroids is about 85% or more of total microparticles in each of the microparticle-localized microspheroids.
   B18. The composition of any one of embodiments B4-B15, wherein the majority of the microparticles in each of the microparticle-localized microspheroids is about 90% or more of total microparticles in each of the microparticle-localized microspheroids.
   B19. The composition of any one of embodiments B4-B15, wherein the majority of the microparticles in each of the microparticle-localized microspheroids is about 95% or more of total microparticles in each of the microparticle-localized microspheroids.
   B20. The composition of any one of embodiments B4-B15, wherein the majority of the microparticles in each of the microparticle-localized microspheroids is about 99% or more of total microparticles in each of the microparticle-localized microspheroids.
   B21. The composition of any one of embodiments B1-B20, wherein the microspheroid sub-region is a microspheroid cap.
   B22. The composition of embodiment B21, wherein the microspheroids comprise an average microspheroid radius r, and: (i) a cap radius a is less than ⅔ of microspheroid radius r, or (ii) a cap height h is less than half of the microspheroid radius r, or (iii) the cap radius a is less than ⅔ of microspheroid radius rand the cap height h is less than half of the microspheroid radius r.
   B23. The composition of any one of embodiments B1-B22, wherein:
the microspheroids comprise an average total microspheroid surface area, and
the microspheroid sub-region is defined by a surface area of about 25% or less of the total microspheroid surface area.
   B24. The composition of any one of embodiments B1-B23, wherein:
the microspheroids comprise an average total microspheroid volume, and
the microspheroid sub-region is defined by a volume of about 15% or less of the total microspheroid volume.
   B25 and B26 (reserved).
   B27. The composition of any one of embodiments B1-B24, wherein:
each of the microspheroids in the plurality of microparticle-localized microspheroids contains a microparticle pattern; and
the plurality of microparticle-localized microspheroids contains about 1,000 to about 100 million or more different microparticle patterns.
   B28. The composition of embodiment B27, wherein the plurality of microparticle-localized microspheroids contains about 1,000 to about 100 million different microparticle patterns.
   B29. The composition of embodiment B27, wherein the plurality of microparticle-localized microspheroids contains about 1,000 to about 10 million different microparticle patterns.
   B30. The composition of embodiment B27, wherein the plurality of microparticle-localized microspheroids contains about 1,000 to about 1 million different microparticle patterns.
   B31. The composition of embodiment B27, wherein the plurality of microparticle-localized microspheroids contains about 1,000 to about 500,000 different microparticle patterns.
   B32. The composition of embodiment B27, wherein the plurality of microparticle-localized microspheroids contains about 1,000 to about 250,000 different microparticle patterns.
   B33. The composition of embodiment B27, wherein the plurality of microparticle-localized microspheroids contains about 1,000 to about 100,000 different microparticle patterns.
   B34. The composition of embodiment B27, wherein the plurality of microparticle-localized microspheroids contains about 1,000 to about 10,000 different microparticle patterns.
   B35. The composition of embodiment B27, wherein the plurality of microparticle-localized microspheroids contains about 1,000 to about 5,000 different microparticle patterns.
   B36. The composition of any one of embodiments B1-B35, wherein the microparticle-localized microspheroids are isolated, and/or are separated or purified from other components.
   B37. The composition of any one of embodiments B1-B36, wherein the microparticle-localized microspheroids comprise a biological entity.
   B38. The composition of embodiment B37, wherein the biological entity is chosen from a cell, nucleic acid, peptide, polypeptide, saccharide, polysaccharide, fatty acid, lipid, and cholesterol.
   B39. The composition of any one of embodiments B1-B38, wherein the microparticle-localized microspheroids comprise a reagent.

C1. A method for differentiating a microspheroid from other microspheroids in a population of microspheroids, comprising:
(i) capturing an image of the microparticles in the sub-region of an individual microspheroid for multiple microspheroids in the plurality of hardened microparticle-localized microspheroids of any one of embodiments B1-B39, thereby capturing multiple microparticle images;
(ii) differentiating one microparticle image from other microparticle images within the multiple microparticle images, thereby identifying a differentiated microparticle image; and
(iii) differentiating, after (ii), an individual microspheroid from other microspheroids in the population of microparticle-containing microspheroids according to the differentiated microparticle image.
   C2. The method of embodiment C1, wherein the image substantially is a two-dimensional image.
   C3. The method of embodiment C1 or C2, wherein the image is identified by optical detection.
   C4. The method of embodiment C3, wherein the optical detection is by microscopy.
   C5. The method of embodiment C4, wherein the microscopy is bright field microscopy.
   C6. The method of any one of embodiments C₁-C₅, wherein the microparticle images differentiated in part (ii) are for microspheroids in which 50% or more of the microparticles are not overlapping.
   C7. The method of any one of embodiments C₁-C₆, wherein:
each of the microparticles is defined by a microparticle radius; and
the microparticle images differentiated in part (ii) are for microspheroids in which 50% or more of the microparticles are spaced a distance greater than the microparticle radius from other microparticles.
   C8. The method of any one of embodiments C3-C7, wherein:
each microspheroid includes a visible microspheroid edge defined by the optical detection; and
the microparticle images differentiated in part (ii) are for microspheroids containing no microparticles contacting the visible microspheroid edge.
   C9. The method of any one of embodiments C1-C8, wherein capturing multiple microparticle images comprises consistently orienting the sub-region orientation of each of the microspheroids.
   C10. The method of embodiment C9, wherein:
the microparticles are magnetic; and
the sub-region orientation of each of the microspheroids is consistently oriented with a magnet.
   C11. The method of embodiment C9 or C10, wherein:
the microparticle image is captured by a detector element; and
the sub-region is oriented towards the detector element.
   C12. The method of embodiment C11, wherein:
the microparticle image is captured by a microscope comprising a microscope objective; and
the sub-region is oriented facing the microscope objective.
   C13. The method of any one of embodiments C1-C12, wherein:
the microparticles comprise a detectable feature; and
the detectable feature is detected.
   C14. The method of embodiment C13, wherein:
the detectable feature is a fluorophore; and
fluorescence emitted by the fluorophore is detected.
   C15. The method of embodiment C14, wherein the microparticle image is captured by a process comprises detecting the microparticles by microscopy and by fluorescence.
   C16. The method of any one of embodiments C1-C15, comprising:
capturing microparticle images and detecting a detectable feature in the population of microparticle-containing microspheroids at a first point in time;
capturing microparticle images and detecting the detectable feature in the population at a second point in time;
differentiating individual microparticle images each associated with an individual microspheroid from the microparticle images captured at the first point of time and the second point of time; and
detecting the presence or absence of a change in the detectable feature between the first point in the time and the second point in time for individual microspheroids according to the individual microparticle images.
   C17. The method of embodiment C16, wherein the detecting comprises detection by one or more of microscopy, nucleic acid sequencing and spectrometry.
   C18. The method of embodiment C17, wherein the spectrometry is mass spectrometry, or Raman spectrometry, or mass spectrometry and Raman spectrometry.
   C19. The method of any one of embodiments C1-C18, comprising segmenting the microparticle images of part (i), thereby generating the microparticle images of part (i).
   C20. The method of embodiment C19, wherein the segmenting comprises a Hough circle detection process.
   C21. The method of any one of embodiments C1-C20, comprising cropping the microparticle images of part (i), thereby generating the microparticle images of part (i).
   C22. The method of any one of embodiments C1-C21, comprising optically encoding the microparticle images of part (i), thereby generating optically encoded microparticle images.
   C23. The method of embodiment C22, comprising comparing one of the microparticle images of part (i) to other comparison microparticle images of part (i).
   C24. The method of embodiment C23, wherein the comparing is according to an iterative image-by-image process.
   C25. The method of embodiment C23 or C24, comprising rotating the one microparticle image in increments.
   C26. The method of embodiment C25, therein the one microparticle image is rotated in one-degree increments.
   C27. The method of any one of embodiments C22-C26, comprising generating a comparison score for the one microparticle image, thereby generating comparison scores.
   C28. The method of embodiment C28, comprising associating the one microparticle image with a microspheroid and differentiating the one microparticle image from the microparticle images associated with other microspheroids based on each of the comparison scores.
   C29. The method of any one of embodiments C1-C28, comprising generating coordinates for the microparticles within a microparticle image and generating a nearest neighbor graph containing the coordinates.
   C30. The method of embodiment C29, wherein the coordinates are two-dimensional coordinates.
   C31. The method of embodiment C29 or C30, comprising comparing a graph for one microparticle image to graphs corresponding to comparison microparticle images, thereby generating a graph comparison.
   C32. The method of embodiment C31, comprising associating the one microparticle image with a microspheroid and differentiating the one microparticle image from the microparticle images associated with other microspheroids based on the graph comparison.

D1. The process of any one of embodiments A1-A48, the composition of any one of embodiments B1-B39, or the method of any one of embodiments C1-C32, wherein the hardened microparticle-localized microspheroids comprise a microparticle pattern diversity of about 1,000 to about 1 million or more individual microparticle patterns differentiable from one another.

D2. The process, composition or method of embodiment D1, wherein the microparticle pattern diversity is about 1,000 to about 500,000 individual microparticle patterns differentiable from one another.

D3. The process, composition or method of embodiment D2, wherein the microparticle pattern diversity is about 1,000 to about 250,000 individual microparticle patterns differentiable from one another.

D4. The process, composition or method of embodiment D3, wherein the microparticle pattern diversity is about 1,000 to about 100,000 individual microparticle patterns differentiable from one another.

E1. A kit, comprising a component utilized in a process for preparing a plurality of hardened microparticle-localized microspheroids.
E2. The kit of embodiment E1, comprising a microparticle component.
E3. The kit of embodiment E1 or E2, comprising a component for forming droplets.
E4. The kit of embodiment E3, comprising an aqueous solution, oil, surfactant and/or fluidic device.
E5. The kit of any one of embodiments E1-E4, comprising a hardening component.
E6. The kit of any one of embodiments E1-E5, comprising a magnet.
E7. The kit of any one of embodiments E1-E6, comprising instructions for conducting a process of any one of embodiments A1-A48 and D1-D4.
E8. The kit of any one of embodiments E1-E6, comprising instructions for preparing a composition of any one of embodiments B1-B39 and D1-D4.
E9. A kit, comprising a component utilized in a method for differentiating a microspheroid from other microspheroids in a population of microspheroids.
E10. The kit of embodiment E9, comprising a magnet.
E11. The kit of any one of embodiments E1-E10, comprising an optical device.
E12. The kit of any one of embodiments E1-E11, comprising an imaging device.
E13. The kit of any one of embodiments E1-E12, comprising a reagent and/or a buffer.
E14. The kit of any one of embodiments E9-E13, comprising instructions for conducting a method of any one of embodiments C1-C32 and D1-D4.

### EXAMPLES

The examples set forth below illustrate certain implementations and do not limit the technology.

### Example 1: Materials and Methods

Microparticles (FAM-2052-2, obtained from Spherotech) were added to a 5% w.w. PEGDA solution containing 0.15% w.w. lithium acyl phosphinate prior to micro-sphere formation. A 10 uL volume of microparticle suspension (at 1E8/mL) was added to 100 μL of polymer solution also containing bacterial cells at a concentration, which would result in final cell occupancy of 1 per 10 droplets. Polymer solution with cells and microparticles was encapsulated into droplet microspheroids using a microfluidic droplet generation chip with HFE7500 oil containing 2% of fluorinated surfactant. An emulsion containing the microspheroids formed in a 1.5 mL Eppendorf tube was suspended above a 30 mm diameter neodymium magnet for 30 minutes to effect microparticle sedimentation to the bottom of the microspheroids in the tube. The emulsion was then illuminated for 5 minutes using 10 mW/cm², 405 nm light to form hydrogel microspheroids having fixed microparticle positions.

Excess oil was removed by pipetting, the emulsion was then broken using perfluoro octanol and PBS was added to increase the volume to 1 mL. The resulting hydrogel microspheroid suspension was then processed using centrifugation and the procedure of adding PBS and centrifuging was repeated several times to ensure microspheroids were washed. Bacterial culture media was then added to disperse microspheroids and they were cultured in a plastic petri dish for 30 minutes before they were imaged on Zeiss Axiovert fluorescence microscope using 10× objective and 50-100 ms exposure for fluorescence readout. A 30 mm diameter neodymium magnet was used to provide additional force to align micro-spheroids with the microparticle sub-region facing the microscope objective. From images captured, micro-spheroids were segmented, cropped and saved in silico. The microspheroid suspension was then cultured overnight in a plastic petri dish, after which images of growing cells within micro-spheroids were captured using the same imaging procedure. Cropped microspheroid images from the first and second imaging experiments were matched to reveal cell heterogeneity within the encapsulated bacterial population.

### Example 2: Optical Detection of Microparticle Patterns in Microspheroids

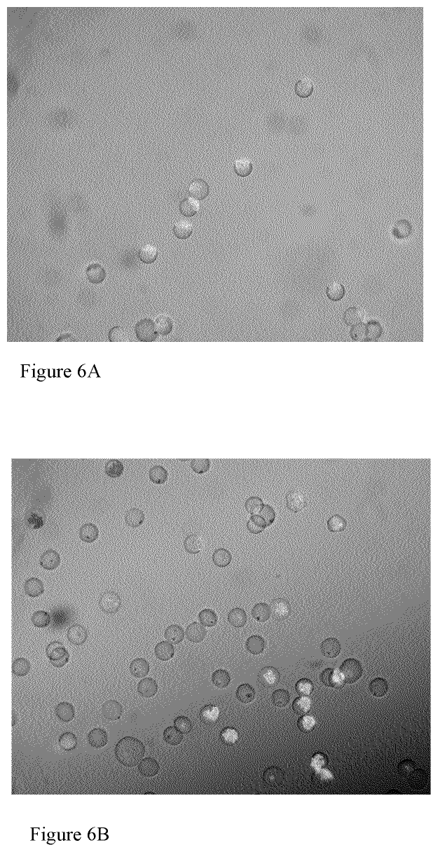

Microparticle patterns in microspheroids prepared by methodology described in Example 1 above were differentiated and associated with particular microspheroids. It was determined that orienting the sub-region containing microparticles in the microspheroids with the microparticle pattern facing towards the microscope objective resulted in consistency between recorded distances between microparticles (FIGS. 6A-6B, addressed herein). It also was determined that fluorescent and magnetic microparticles enhanced image resolution and segmentation.

FIGS. 6A-6B illustrate use of a magnetic field to align microspheroids for efficient imaging. FIG. 6A illustrates an example of an image captured without using an external magnetic field to align the microparticle cluster towards the objective. Microparticles at the visible edge of the micro-spheroid make it impossible to discern their unique pattern. FIG. 6B illustrates an example of a microspheroid image captured with a magnet under the petri dish to align microparticles for imaging readout. In this approach there were fewer microparticles contacting the visible edge. The images show merged bright-field and fluorescence images, where fluorescent microparticles facilitate distinguishing the microparticles from cells and debris. Median microspheroid diameter was 45 micrometers.

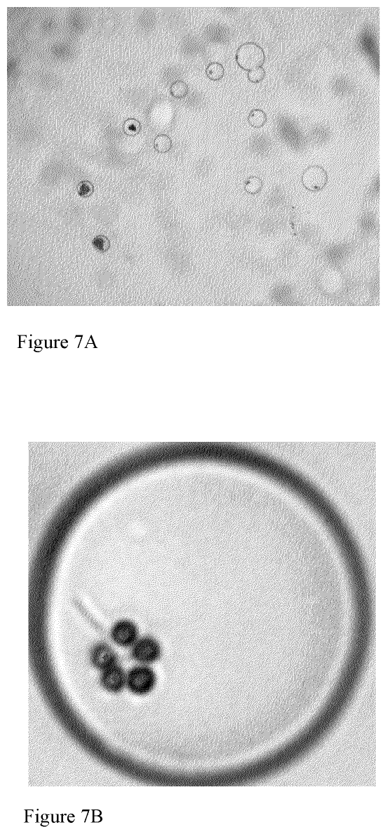

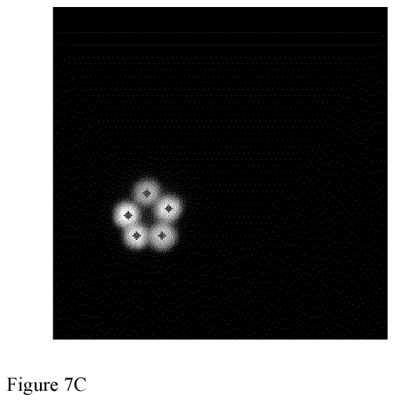

FIGS. 7A-7C illustrate microspheroid segmentation and optical encoding processes. A microscopy image (FIG. 7A) is segmented using a Hough circle transform. Then cropped single microspheroid images are analyzed by detected microparticle positions within the microspheroids from bright-field (FIG. 7B) and microparticle fluorescence (FIG. 7C) images.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A process for preparing a plurality of hardened microparticle-localized microspheroids, comprising:
(a) providing a population of microparticle-containing microspheroids in which at least a subpopulation of the microspheroids contain a plurality of microparticles smaller than the microspheroids;
(b) exposing, after part (a), the population of microparticle-containing microspheroids to sedimentation conditions that localize the microparticles to a microspheroid sub-region, thereby generating a plurality of microparticle-localized microspheroids containing the particles in a sub-region of each microspheroid; and
(c) exposing the plurality of microparticle-localized microspheroids to hardening conditions, thereby generating a plurality of hardened microparticle-localized microspheroids.

2. The process of claim 1, wherein the microspheroids in part (a) are chosen from droplets, hydrogel beads or two-component microspheroids.

3. The process of claim 1 or claim 2, wherein the microspheroids in part (a) comprise an interior comprising a first component and an exterior comprising a second component.

4. The process of claim 3, wherein the second component is a cross-linking polymer.

5. The process of any one of claims 1-4, wherein the hardened microparticle-localized microspheroids are about 30 micrometers to about 100 micrometers in diameter.

6. The process of any one of claims 1-5, wherein the microparticles are of a first density and the microspheroids in part (a) are of a second density less than the first density.

7. The process of any one of claims 1-6, wherein the microparticle-containing microspheroids in part (a) on average contain about 5 microparticles to about 100 microparticles.

8. The process of any one of claims 1-7, wherein:
the microparticles are of a first density and the microspheroids are of a second density less than the first density; and
the sedimentation conditions in part (b) comprise sedimenting the microparticles by gravity for a period of time to the sub-region of the microparticle-containing microspheroids.

9. The process of claims 1-8, wherein the sedimentation conditions comprise exposure of the microspheroids to centrifugation.

10. The process of any one of claims 1-9, wherein:
the microparticles are magnetic; and
the sedimentation conditions in part (b) comprise applying a magnetic field to the microparticle-containing microspheroids that sediments the microparticles to the sub-region of the microspheroids.

11. The process of any one of claims 1-10, wherein:
the microspheroids comprise a thermally responsive hydrogel polymer, and
the hardening conditions in part (c) comprise cooling the microparticle-localized microspheroids to a temperature below a sol-to-gel transition temperature.

12. The process of any one of claims 1-11, wherein:
the microspheroids comprise a cross-linking polymer; and
the hardening conditions in part (c) comprise exposing the microparticle-localized microspheroids to cross-linking conditions.

13. The process of any one of claims 1-12, comprising isolating the hardened microparticle-localized microspheroids.

14. The process of any one of claims 1-13, wherein the hardened microparticle-localized microspheroids comprise a biological entity.

15. The process of claim 14, wherein the biological entity is chosen from a cell, nucleic acid, peptide, polypeptide, saccharide, polysaccharide, fatty acid, lipid, and cholesterol.

16. The process of any one of claims 1-15, wherein 80% or more of the microparticles in the hardened microparticle-localized microspheroids are disposed in the microspheroid sub-region.

17. The process of any one of claims 1-16, wherein, for a majority of the hardened microparticle-localized microspheroids, the majority of microparticles are disposed in a monolayer in the microspheroid sub-region of the hardened microparticle-localized microspheroids.

18. The process of any one of claims 1-17, wherein the microspheroid sub-region is a microspheroid cap.

19. The process of claim 18, wherein: the microparticle-localized microspheroids are defined by an average microspheroid radius r, and: (i) the microspheroid cap comprises a radius a less than ⅔ of the microspheroid radius r, or (ii) the microspheroid cap comprises a height h less than half of the microspheroid radius r, or (iii) the microspheroid cap radius a is less than ⅔ of the microspheroid radius rand the microspheroid cap height h is less than half of the microspheroid radius r.

20. The process of any one of claims 1-19, wherein the microspheroid sub-region is of a surface area of about 25% or less of the total microspheroid surface area.

21. The process of any one of claims 1-20, comprising, prior to part (a):
contacting one or more microspheroid-forming components with a plurality of microparticles; and
forming microspheroids under formation conditions in which multiple microparticles are incorporated within microspheroids in a population of formed microspheroids, thereby providing the population of microparticle-containing microspheroids in part (a).

22. The process of claim 21, wherein the microspheroid-forming components are droplet-forming components and the microparticles are incorporated into formed droplets.

23. A composition, comprising a plurality of hardened microparticle-localized microspheroids, obtainable by a process according to any one of claims 1-22.

24. A composition, comprising a plurality of microparticle-localized microspheroids, wherein each of the microspheroids comprises a plurality of microparticles within a microspheroid sub-region.

25. The composition of claim 24, wherein the microparticle-localized microspheroids are about 30 micrometers to about 100 micrometers in diameter.

26. The composition of claim 24 or 25, wherein the microparticles are about 1 micrometer to about 5 micrometers in diameter.

27. The composition of any of claims 24 to 26, wherein the microparticles are of a first density and the microspheroids are of a second density less than the first density.

28. The composition of any of claims 24 to 27, wherein the microparticles are magnetic.

29. The composition of any of claims 24 to 28, wherein the microparticles are optically detectable.

30. The composition of any of claims 24 to 29, wherein a fluorophore is linked to the microparticle.

31. A method for differentiating a microspheroid from other microspheroids in a population of microspheroids, comprising:
(i) capturing an image of the microparticles in the sub-region of an individual microspheroid for multiple microspheroids in the plurality of hardened microparticle-localized microspheroids in a composition of any one of claims 23-30, thereby capturing multiple microparticle images;
(ii) differentiating one microparticle image from other microparticle images within the multiple microparticle images, thereby identifying a differentiated microparticle image; and
(iii) differentiating, after (ii), an individual microspheroid from other microspheroids in the population of microparticle-containing microspheroids according to the differentiated microparticle image.

32. The process of any one of claims 1-22, the composition of claim 23 or any of one of claims 24 to 30, or the method of claim 31, wherein the hardened microparticle-localized microspheroids comprise a microparticle pattern diversity of about 1,000 to about 1 million or more individual microparticle patterns differentiable from one another.

33. A kit for preparing a plurality of hardened microparticle-localized microspheroids, comprising a microparticle component; a component for forming droplets comprising an aqueous solution, oil, surfactant and/or fluidic device; and a hardening component.

34. A kit, comprising a component utilized in a method for differentiating a microspheroid from other microspheroids in a population of microspheroids.
